SEQUENCE LISTING (9 sequences)

SEQ ID NO: 1 - DNA, 2158 bp, Murine
ctgtgatattccttcttcttcccgtctcaattaaaaatgatctcttggctgggcacagtggctcatgcctgtaatcccagcactttgggaggcccatgcaggtggatcacctaaggtcaggagttcagaccatcctggccagcatggtgagaccccatctctactaaaaatacaaaaactagccaggcatggtggcgggcgcctgtaatcccagctacttgggagactgaggcaggagaatcgcttgaaccagggaggcggaggttgcagtgagctgagatcaggccactgcactccagcctgggcgacagagtgagactccgtttcaaaaaaataaaactgatttctggaagatcatctaatagaaacattcagcatcttggtctatttctctatcctccccgcactaacatctgaaaatggcacctacctattcactcattaacctcttggaaaatgacttctgcccaccctacctgctttatactaaaactgatctccagtgtcacttaacctgttaagggccagtaatctcccagtaacctggctgtgactgccttctttaacctccactcttggtgctcctgttatgcctgcttgtcaccacagcctagccctagagattctatctttccaatgccttcccttctatcctctcctaaccatctattattctgtctattactcctaaatcagttcctgatttctgattatctggatgatgagcagcatgcttctaacaaatctttctctctccagcctctttcccactccaactgaaaaccatgggttttccatcatccttttctccaaaactcttatttccaagtcaattaagcactaactccccaatctgatattcaattacttccgcagttttaacacaattccacatgtaacggcatcggtcagcattatgtcccactgctattccaagcataccctgtgctccagccacagcagtctgcttattttgcacgaaacacatggcgtgcttttgcacctccaggctttggttcaccccctttttttttaatccacctggattatgtttcccctccgtctctactattctaaatcccatcctcccctaatttcacttgccacgctatcttcactaaccttctaccaaacgtattataattatttgtgcggttatcatatttccacactggattcttaattctgctaaaacgaaatactgtaatgcctttgatgagccttccatgtgccagccatgtgctaagtgagctacatgcattatctcatttaatcctcaaatcgccatctaggtcaggactctcccatttaacattaaacaaactgaggctccgagagattaagtagcttgcagctttcacgcagctaattatggtggaatcagattccctccccgaggttcaacttgtattagttagggtagccagcataatatattcgccagctacgctgcactcaacctgtgtgtgccggatcagcatgttaaaaacggtgtctgcaagtagaccaagggaagaggggctgcgggggcgggaattgagacagcaatgaattaggatggcgggattggcggtgatgtttccaaatttccactgttgggttgtagagcttctgaaataagcatttaaaatgtatagttactaaacaagattgcatctacccttggtctaattgttatgtgtaggttagttttgtctccctgattttattgtcatcccccagggattgtttctgttctcccgcgttcccagagctttcggcgcattgctcaacacacggtgtgcgtacttgttgattttaatgtatgtgtgtttgcatatgtgcacagcagctgggcggaggacgtgggtgtgggatgcactgtgcattccttctcctcactcttgttcctttgagcagctgcagcctggatgctttcccttaggggtaggggagaggcagaaccgctgaccctttaacaagttatccctcacagaacaggtgtctcccccgactctcggtggagagggatggctggatacttagtgacatcagccacgagccaatgggaagtcgggctcctgcacggcgcggctcggctcatgcccccgggcgcggggcacacaggccggccggcagccgctgggaaataggcccccgggggcggtggcggcggcgg

SEQ ID NO: 2 - DNA, 2017 bp, Murine
cggccgcggtggagtctccgctgctcacgctgcccgccgcggcttcagcggcggcgccgttccctggccgcgcggctcccagacgcacggttcgggcggctcccggcgcggcgcggctgccccggcgttcgggtccccgtggccccgcggctcccgccccgggctgcagggcgcacaggtgggccggtggccgctgggagataggccccccgggggcagcggcggaccatggcgcggagacctggcgtgccggctgcctacggggacgagttctccttcgtcagcccgctggtgaaatacctgctcttcttcttcaacatgctcttctgggtaatctccatggtcatggtggccgtgggtgtctatgccaggctaatgaagcacgcagaagcagccttggcttgtctggcggtggaccctgctatcctgctgatcgtggtgggcgtcctcatgttcctgctcaccttctgcggctgtatcggatcccttcgggagaacatctgccttctgcagacgttctccctctgcctcaccatcgtgttcctgctgcagctggctgctggcatcctgggcttcgtcttctcagacaaggctcgggggaaagtgagcgagatcatcaacaatgctatcgtgcattatcgagatgacctggatctgcagaacctcatcgactttggccagaagaagttcagctgctgtggagggatctcctacagagactggtctcagaatatgtacttcaactgctcggaggacaaccctagccgggagcgctgctctgtgccttattcctgttgcttgcccacccccaaccaggcagtgatcaacacgatgtgtggccaaggtatgcaggctctggactacttggaagctagtaaggtcatctacaccaatggctgtattgacaagttggtcaactggatacacagcaacctgttcctgcttggtggtgtggcactaggcctggccatcccccagctggtgggaatcctgctgtcccaggtcctcgtgaatcaaataaaagatcagatcaagctacagctctacaaccaacagcaccgagctgacccgtggtactgagaatccttcctgcgcctccttaccatggcgacgagcatctcacggaccagcagcagagggtgctgagagcacccagtgcagatttggattccagcccccaaatgagggcccagtggaaagaagcaagctccagaaggcaggacacagatggctcaggtctccgaaagatgtgcctcatctccccatcctagcctcagcattgtccgaggtttatttcatgctgtttgtaaccttgaacatttgagtttgtgtttttagtttcctctgggcagaggtgtggaaactgcagcggcacagaaagctgtaggattactagctgcatactgaggcacaacagacagctctgttagggctgctgctgagctaggtggacagagtctgctgccagggagtcctggctgtgcacagatactggcagccctgcaggagtccagttggcacaattccacctccagagaggctgggagtgcagcgggcagagcagcaggagcctagggaacctaggggtcggatcgggaccgcagggcacatgggggataggagtgaagggggtatgtttaccaaatgcttgcctagccgcactcccaggtagtcagagtgagctatatcctgctcctccctcgtctccatggaaacaaggctgcaagggcaaggggtataacagcagccaaattcatcctcacccgctctgtaaaatgtttggaaccgtggtccctaaattctgcgtgccggactaagccatacatgcccttgaattcctctctgtctggccttcccgccccagcaagtggggttttctttaacttggcagaggagtggcaccaccacccacccgcatcccctgcaccagaactcagtgtttctacagtgtaagaatcgggaatctggcagaaatggcaataaaagtttgttgacatggaaaaaaaaaaaaaaaaaa

SEQ ID NO: 3 - protein, 283 aa, Murine
MARRPGVPAAYGDEFSFVSPLVKYLLFFFNMLFWVISMVMVAVGVYARLMKHAEAALACLAVDPAILLIVVGVLMFLLTFCGCIGSLRENICLLQTFSLCLTIVFLLQLAAGILGFVFSDKARGKVSEIINNAIVHYRDDLDLQNLIDFGQKKFSCCGGISYRDWSQNMYFNCSEDNPSRERCSVPYSCCLPTPRQAVINTMCGQGMQANDYLEASKVIYTNGCIDKLVNWIHSNLFLLGGVALGLAIPQLVGILLSQVLVNQIKDQIKLQLYNQQHRADPWY

SEQ ID NO: 4 - DNA, 1919 bp, Human
ctcggctcatgcccccgggcgcggggcacacaggccggccggcagccgctgggaaataggcccccgggggcggtggcggcggcggggccatggcgcggagaccccgggcgccggccgcctccggggaggagttctccttcgtcagcccgctggtgaaatacctgctcttcttcttcaacatgctcttctgggtgatttccatggtgatggtggctgtgggtgtctacgctcggctaatgaagcatgcagaagcagccctagcctgcctggcagtggaccctgccatcctgctgatcgtggtgggtgtcctcatgttcctgctcaccttctgtggctgcattgggtccctccgcgagaacatctgcctcctgcagacgttctccctctgcctcaccgctgtgttcctgctgcagctggccgctgggatcctgggcttcgtcttctcagacaaggctcgagggaaagtgagtgagatcatcaacaatgccattgtgcactaccgagatgacttggatctgcagaacctcattgattttggccagaaaaagtttagctgctgtggagggatttcctacaaggactggtctcagaacatgtatttcaactgctcagaagacaaccccagtcgagagcgctgctctgtgccttactcctgttgcttgcctactcctgaccaggcagtgatcaacactatgtgtggccaaggtatgcaggcctttgactacttggaagctagcaaagtcatctacaccaatggctgtattgacaagttggtcaactggatacacagcaacctattcttacttggtggtgtggctctaggcctggccatcccccagctggtgggaattctgctgtcccagatcctagtgaatcagatcaaagatcagatcaagctacagctctacaaccagcagcaccgggctgacccatggtactgagaatccatcctgcacctcctcaccatggaaactggcaagcctcataaacgaacagcagtgggtgctgaaagcagcaccaaatggagatttggattccagccccccagtgacagcccagtgggaagaagcaaactccagatgggcagaaggcagggtgcacaggtggctccagtctcaggaggatgcgcctcctctcccccatcccagccctcagcattgtgccagagtgatacccttaagtgtttgggtttatgttttcagttttgtttgggaaacagcagttgcacagagagttgggggtactgctgctgccttttcaccgaggcactgccaccaccagctctagcagggatgctcctgagcttggcggacatacttagatcctaacgtgccagtgagacctggctgtggagagtagcactggcagccctgcctggactccacttggcatgataccagctccagaagggaagggagtggagcaggcagtgaggagagagcctgggggtcggctggggacagccgtatgtgctaggtaggagtggagggagatatgtttaccaaatgcctgtcctgccatcctcccaggtagtcagagtgagctacatcctgccccaccttcatttccatggaaacatggcagctaggacacggggtacaacagcagccaaattcttccccacctcccttacttcgaaaaaaaagtttggaaccctggtccctatactctgcagtcagaagtgggactgagccatacatgcccttgaattcctccctgtctggccctccctctccagcaagcagggttttctttaacttggcagtgtgcagaggagaagtggtaacacccccaccccattcccctgcatcggagctcagtattcctacagggtaagaggtaggaatcttgctgggacgaggggagccagaagtggcaataaaagcgtgttgacctgggctaaaaaaaaaaaaaaaaaa

SEQ ID NO: 5 - protein, 283 aa, Human
MARRPRAPAASGEEFSFVSPLVKYLLFFFNMLFWVISMVMVAVGVYARLMKHAEAALACLAVDPAILLIVVGVLMFLLTFCGCIGSLRENICLLQTFSLCLTAVFLLQLAAGILGFVFSDKARGKVSEIINNAIVHYRDDLDLQNLIDFGQKKFSCCGGISYKDWSQNMYFNCSEDNPSRERCSVPYSCCLPTPDQAVINTMCGQGMQAFDYLEASKVIYTNGCIDKLVNWIHSNLFLLGGVALGLAIPQLVGILLSQILVNQIKDQIKLQLYNQQHRADPWY

SEQ ID NO: 6 - DNA, 1216 bp, Murine
ggggtggaagggtggcccggggtgccccacgcgcgggagccgcactgccgagttttctgggctgacggcactttgggggaatttatcctggggagggacaccccggggacgccgcgtctgcagggggcgttgtagggaggagggtatgccagggaatcagtaaagactgatgggtattctcggggcgtcggaggagggtctgtgttaggctagaaatgtgggtttgagggagcctgttggcccaaggcggacccactgacacattcatgatgggggtcgggggtggcggctgactttcaggcctgggggcctggaggcccggctggcaaagaaagaagaggaaggaaaaacccaaacctctctctccaccacctcagagaggatgggttactggcatcatctcttccttttgcaggcctcatctacccccacctccacccccccaaagtctaggaagaatggcctccaggtgcagatgtctttcccatcccagcactgccccaaactttgtcccctccctgccacccttctcaggaccagacttcacccctggagcctggcaatcaccagtgtctttaaacaggaccccgtgttgggccggagggccaggggtcctttcagaatcttctgcttggggtggaaaaagtattgggctgggactccggacacctggattcccatcccactctgccgttcattgttgacttgaccgacctaagcatgtatgttcatgtctgcaggccttgactccctcccccacaaatccaacccagtgtgatcaggggtggcagaaagactgctgaagctgagaattgagctgaaccagatcatgcaatgccagctcgaccctgtttcctggctagagagtcctggaggtcctggggtgtgagaacatagggacatccttcccccattcacctcctcttctggctcctaccacaccagagctgctgcttctggagcccaccatccttgcccacttcagctggagacctaaagttcagacttgcaggagggaagaattgagtctaaaatatgtttcctaggggagggagcagaacagacatctaagccatttgctggaaaggtttcacagggcccgtggggtgaggtgcagacacaaagcccataagtgctggcctgttgggacaaatgagagaaatcccatagggtggtgatgacagcgcactcagccatcctactcctggggaaaatg

SEQ ID NO: 7 - DNA, 11922 bp, Murine
atcattagtgatcatattaaatattaataataatattaaagacttaatcttgattttaatgtagcctgtatctgtggctctagatccctggctgattattctttaggcttgtaggttctcaagcttggctgcacattgaaataacctggaacacattcagaaatactgttgcctaggtcccaatgccaaagattctgatcgacttggtttgggatgcagcctaggcattagcagttttttaagcctctcccccaacaaccctaagtcccaggcgattctaatggcagccaaagttgagcatgtgctttcagctataaacgctcttgtccctccaccatatccacgcatggattaagggtcctcagtgtaccatctgcatagcagcagatgaattggatctggacagtgtattagtttcctgttgctgctgtaacaaatgaccgcaaacttagtggcttaaaacaacacacattaattttcttatatttctggtggccagaaggccaaaacaggtttcaccgggctaaaatcaaacagatctcgctgggctaaaatacagcagccccgcattccttcgggaagtgctaggaagagctcgcctccttgcctgctccagctgttagaggcagccctcattcctccatccgtggcaccacagcagtccagcctctgcctccatcaccgtctccttctctgactctggccctcttgcctccttcttagaaggtcttttatgattatactgagtcctcagggataatttccctagctcaaaattcttaatcacatttgcaaagttcctttggccatgaaaagtaacacaatcacaggttccagcaattaagacgtggacatctttgaggggacattattctctccaccatagatagagacagagcaactgctgtcattacaggcccataaatacagttaacttactctatgatatgtggcatctctttactttgcccattttttcaacttttattttaggttcagaggtacatgtgcaggtttgttatataggtaaattatgtgtcatggggtgatatttcataacccaggtaataagcataagtagttttccgtcctctccttcctcctacccttcactctaaagtacatgtggccatgtgtactcaatgtttagttcccacttacaagtgagaacacatggagtttggttttctgttcctggattaatctgcttaggataatggcctccagctgtatccatgttgctgcaaaggacatgatttcattcgtttttatggctgcgtagtattccatggtatatatgtaccacattttcttttcttttttttcttttgatatagagtcttgctctgtcgctaggctggagtgcagtggcacgttctcagctcactgcaacctccgcctcccgggttcaagtgattcttctgcctcagcctcctaagtagctgggactacaggcatgtgccaccatgcccagctaatttttgtatttttagtagagatggggtttcaccatgttggccaggatggtctcaatctcttgacctcatgatccacctgcctcagcctcccaaagtgctgggattacaggcatgagccactgcacccggccccacattttctttttctttctttttttttagagatgaagtctcactctgttgcccaggctggagtgcagtggcatgatctcggctcactgcaacctccgcctcccaggttcaagcgattctcctgcctcagcctcccaagtagctgggactacaagtgtgtgccaccatgtccagctaatttttgtatttttagtagagatggggtttcaccatgttggccaggctggtcccaaactcctgacctcgtgatccacccgctttggcctaagtgctgggattataggcgtgagccaccgtgtttggcctgaccccacattttctttttcccgtactgttggtgggcatttaggttgattccatgcctttgctattgtgagtagtgcttgtttggcctttcttgttttagctgtgctacgatttaatcagataccatctttgtggaaaattatacaccctggtgaaaatttgacatgaaaaacaatgagagcctggaagcaacagaaatcaagagcagatacacatttttcaatagttcagtaaaatgacactcagaactgtaattggctgcagtgggttttaaagggcagacagagatgctcactgactctggtggaggactggccaccaggcctggggaatgactggcatctctgtcatgtagtttccctgcctcccccatgctccgtgagcctggcagtgctgtgggacaaggcgccagctggcacctgcagctctgctagcagctcccaccctcctggacaaagagtatgaaggccagcaaataacactttgggccctgagttagaggcatggtcaaatgatgtgaggtgggaagagaccggggccagctgagtgtggtgactcacacctgtaatcccagcactttgggaggctagggtgggaggatcaattgagcccaggaggtcaaggctgaaatgagctattatcatgccactgcactccagcttaagcaatagcacaagaccctcctccctcaccccgcaaaaaagagggggctgtatatacaggcagagaagagttctggaagacgtggttttagataaaggtttgccatttactgctgaatgcacctgccctcccctgccagatggcagaggaccttcaacaaggaggcctacagggaaaaatgagggactcatctgaccctattacctgttctgggggcctagctcccttcttccaccaaagccccatggtggctctttccagctgctttccgtcttgcaggacagtggtgtctagccagccatctccttccccactgagttggtcaagaccctgctgctgagaactctctcctgccagagcttggccccatctgttgccatggcaacagccagctgctgcttagcaccagcctggagactgggcacaaaaaaagggtggccaagggaagtaggcactggggctctcctgcacccctggaggcctgggggctagggggtagataaggcctctttggggtctcctattagtctctgatgggtttgttctctcagggagaagccaggaaatttgaaagatctaaactcctctctattcctccacactggaatcagagtttgtgaagataaaggttcctttgccaaggcagttccagtgcttacatacatgcactcacacacagtccttcacattggagtgcactttctttttgtacttgacatgatattgacctctaagagggttagagagctgggagtatgcaaccgcccaggcctgctttaggtataaaacctgtcctttgatgcagatagattcgaattcaaaacttttccatttatgtgtgaccctgggcaagttaactattaataatgatagataatatttattgtcaggcattgtgttaaattatttccatgcattattttagttggttcttacagtaatcttgtcagttgtttgttcttacttcccatcttcagatacagaaactgaggctcagaaatttgccctatgtgtacccaagtttgactcgctctgaagactgtgtcctttttaattttaacttcaatttattagagacggggcctcactctggcacccaggcttcagtgccatggcacagtcatggctcactgcagccttgcactcctgggctcaaaccatcctcctgcctcagcctgctgagtagctgggactacaggtgcacaccactgcacccagctaattattattttgtgtgtgtgtctagagacaagctctcactatgttacccaggctggtctcaaactcctgacctcaagttgattctcctgccttggcctcccaaaatgcccagcctgaagcccatattcttaatcactcttcagtattaaccttggattgccttaatttcctcatccgtaaaataggcataatgtctgcctcacaacctaattgtgttacgagcactgaatgaataatgaaaaatatgatgtgggtaaaagtgtctcatggaggtaaagccctgacagattctatcagcagaatcactggggaggtgaaagatgctcacctttgggtcccaggaaagcagtccttctcctgctctcctaggtttctcatggcattcagggctctctggtttccagaatctcagggtctggctcttttcctgtctccacaggtgatttccatggtgatggtggctgtgggtgtctacgctcggctaatgaagcatgcaggtgagctgtagctctccctccctgcccccaccttgtgccatgcccctttcccctctgcctccttcagcctggccctcacacataagctatggggctaaaggaggcagcctccctggagccaaatcttcatggtctacgtaggtgtcagacccccccaggccagtgcccctggatttccctgagcccctctgagtcttccaggatatgcctacatttgaggcttaggaagtgtgtgagaaagaaggtaggcccctgggagcagagagtcatcaagggctttagcccctccctagctctgtcctggagaaacccaaccaccattcctatttcaccacacaagtcaccaggaaacagaatctgcaaagctggtgagcctaatgccattccaattcttttgcggagcgaaagcagcccctccttaagcccaggaataaaggcttttatttctttagttcctcaaattaaatgccacaacccttctgggccctttttggttctccccatgggacaggggtactgcctccccttaaccttcatccaaccataatggagattcccccaccaggggaaggagaacagtttggggcaccctcccaccccatccaggggttcctcagtgggcaccaggctcaggctgagggccggctccttttccagaagcagccctagcctgcctggcagtggaccctgccatcctgctgatcgtggtgggtgtcctcatgttcctgctcaccttctgtggctgcattgggtccctccgcgagaacatctgcctcctgcagacggtgagtggtcaaggccccactggaaagaccctggccctctccccatcatgcctcttagcctcccacggccctttggttgccttgtcctaccctacccccaagcctgtgctgtccacccctcagtgcagagcttagcccgggtgacccagcacagggtggccacccccaagacagtgtcccagaggcagctgcctttctcatgtgtggggcatctgggagaattgcctggcagctcccagcatccccttggccctggaaggggttgctgccatgagtggtcctagacgcctcttcttcctgctgctccacagttctccctctgcctcaccgctgtgttcctgctgcagctggccgctgggatcctgggcttcgtcttctcagacaaggtaacactgggagccaggaggcctcctcaggtgtgacccagagggaggaaggaaggttcctgcccatgtttagcctggttaattaaccacagctccttcccctgagaaacatcagtcgttgaaaagttcattaggggtgaatgaatagcctgtaaactcatcatatttgaagagaattttaagatcataaggtgctttcctctgacagtctgagaagctaacagggaagtaaattatacaaacacaaagttaaaataacttagaaactttaatagcaaatgacagaatcaaactcaataatcaagtggaccactgacaatatgaagagcctcttatttcttttaaaaattgtttattgagataaacagaaggaaggaagggggaagagaacaagattttgttttgaggtttatttttctttctttctttttttttttttttttggttgttgtttttgttttttaagagatgggagtctcactaggttgccaggctggccttgaactcctgggctcaagtgatcctcctgcctccatctcctaagtctctgggattacagtcatgtgccaccatgattggcttaataaaaggttttgaacaatgcttgccatatctggaaatggtctgccattcagtgacagtcctttttaagaaccagaatgtctttcccaaaaggcaaatgattgccaagggcagactacaaaactaaggaaagaaaatttgagaaaagtagtggggcaggaaggaagagactatggtagaaaattatgaaattgggccaggcgcggtagctcatgcctgtaatcccagcattttgggaggctgaggcgggcagatcacttgaggccaggagttcgagtccagcctggccaacatggcgaatccctgtctctactaaaaaacaccaaaaaaattagccaggtgtcaggaggctgaggcacaagaattgcttgaacccaggagaaggaggttgcagtaagctgagatcgcgccactacactccagcctgggcgacagtaaaactgtatcaagaaaagaaaagagaaagggagaggggaggggaggggagaaaattatgaaatcgagagccactgagttaattccgaatttcgtagtcctcctggcttacagtaagatcctgaaatgatgtattaaatagctctgttttgagtctaaagagatgcaattatctagtgtccactagagggcagtgcctcagctcagtaagctactttctgctaaaaaaattaaaaattagtttgtttttatcagtcaacaagtattttgtgataatagcattcagagatagatacacattggtatatcttgaaatgtgtctctgaaaagcttcaacagcaggtccagcatttctcctgcccactggtacctgagggccctgggatgggctttggaactgaaccggctatcttccaccattaggactcggttctgatttcaaggcagactcagttaagaaggatgatccctgtctactgttatttttgctggaaggagcaagttagggattagtcctgtgttctgattccttgcccatgtctccggcaggctcgagggaaagtgagtgagatcatcaacaatgccattgtgcactaccgagatgacttggatctgcagaacctcattgattttggccagaaaaaggtatgggtcagccagtggtctgggggactgtgggtaaaagtgaatgtcatcccaagagatgcctcaccctctatgcctgtggggctcttcattacctgccaggtaatggcttctgggaaggggtttggcaaaaaagcacacgtagcagagtgctttaaatgtacttttaaagacacagaacagtatatatagtaatctactgtgttataaatggttacctacagggggtgaggaactgggcagattcttgaatattacctcttcaaaagtgacattttaggctggtccaaagggagtgagttatctcatttgattgttcacagtcagctacagatccaactccttgttctactctttccccccttctcactgctgcacttgactagactaaaaaaaagaaaattacatttaaaaaattgtgataaaatttacataatgtaaaatttaccatttttaagcatacagatccgtggcatgaagtaccttcaattgttgtgcaaccatcaccaccatctccagaacgctcatctgccccagatgaaactctgtatccatcaaacactaactccccatttcctctcttgaatctctgtatccatcaaacactaactccccattttcctctcttgaaactctgtatccatcaaacactaactccccattttcctctcttgaaactttgtatccattaaacactaactccccattttcctctcctgaaattctgtatccattgaacactaaagccccattttcctctcctccagcaaccaccattctactttctgtctgtaaatttgacaactctaggtacctcaaatgaagggactcacggtattaatgcttttttgagtgggttatttcacttagcatgtcttcaagtttcatctctgttttggcatgtgccacaactgccttccttttgaaggctgaataatattctgttgtatgtatatgccacattttgtttatccattcattcaccagtggagaaaagggttgcttccaccgttcggctactgtgaacaaggctgctatgaatcaaaagttaacaattaggccaggcatggtagcttacatctgtaatcccagcactttgggaggccaaggcaggcagatcatttgaggtcaggagtttgagactagcctggccaacatgatgaaaccctgtttccaccaaaaatacaaaaattagccgggtttggtggcgcacacctgtagtcccagctactcaggaggctgaggtgcgagaatagcttgaatttgggagttggaggttgcagtgagccaagatcatgccattgcactccagcctgggtgacagacagagactccgtctcaaaaataaataagtgaaaaataaaagttaacttttttttttccctgagacggagtcttgctctgttacccaggctggagtacaatggcgtgatcttggcccactgcaacatccgcctcctgggttcaagtgaatctgcctgcctcagcctcctgagtagctgggattacaggtgcctgccaaaccacgcccagctaattcttgtatttttagtaaagatggggttttgcaatattggccaggctggtcttgaactcctgacctcaggtgatctgccctcctcggcctcccaaagtgctgggattaccggtgtgagccacagcacctggccaaaaaagttaacttttaagtaaaagtgcttctgagacaagtcctgagttccccttcctctggtgcaatagacacttgactttgagatccagttggctctcatggagtaatgattgactcacaatattgaagattcctcccctgtgttttctctatcttaaggagaagtttttcagcactgatactgtaattatttcagccaatacttttgatcacttccatttgcctgcaaagccagaaacttctctcaagaagattatagctgagtatgtaaaacaaaatatatgtacaactgtaataccaacaagcacagtggtaagttataacaagatatacgaagtaatagaaattaagaagtgagaatatcttcaggcgaaggcaactgggaagctggagctcaaagtttaaggctgtgttaaaacacaacctaaagttcccagtagaagcttgctatagacttttggtggctctgagaattcccctggtcacctcagtgtgaggggtgggtgattcttaacctctggtgggttttgttgcagtttagctgctgtggagggatttcctacaaggactggtctcagaacatgtatttcaactgctcagaagacaaccccagtcgagagcgctgctctgtgccttactcctgttgcttgcctactcctgaccaggtgagccagcatcctgcctcatttcctcctaggcagcgagctgagtcactttctgatgggggcagctgctactggggatccccatcccctagcttcaccgatcagtcatgtgggacagctgtcaggtgtttttcttcaccccaacctgatgcttctattaacctcatactcatgtagtccacatggagttcagaagggacagatttaagttttaagttcagattcagtacctgtgcaggtttgttatataagtaaacttgtgtcttaggggtttgttgtacagattattccaccacccagagcaaagagacctggatcctgacctctcagaggaagggaagtctgcatttggcaacttccaacccaatatcctccacatatattctctgacaatttaggagtttgggaagggtgggaagcccatcagctaaggccccaaacaaaaagttattggaattacagtcccaattggcttttcaactctttccccaggcagtgatcaacactatgtgtggccaaggtatgcaggcctttgactacttggaagctagcaaagtcatctacaccaatggctgtattgacaagttggtcaactggatacacagcaacctattcttacttggtggtgtggctctaggcctggccatcccccaggtaacttaccctgtgagacttgttggtcccacacactctgtaaagactcctttgttttggggaaggcacctggggatcagcgagggtgtcaggcactgacatggctgaggggtagggaaagggatagtgctggccgcactgggaagatcgagccagggaaaacaaggccatcactcactgctgagtgcccaattccttcttgcctctcccagctggtgggaattctgctgtcccagatcctagtgaatcagatcaaagatcagatcaagctacagctctacaaccagcagcaccgggctgacccatggtactgagaatccatcctgcacctcctcaccatggaaactggcaagcctcataaacgaacagcagtgggtgctgaaagcagcaccaaatggagatttggattccagccccccagtgacagcccagtgggaagaagcaaactccagatgggcagaaggcagggtgcacaggtggctccagtctcaggaggatgcgcctcctctcccccatcccagccctcagcattgtgccagagtgatacccttaagtgtttgggtttatgttttcagttttgtttgggaaacagcagttgcacagagagttgggggtactgctgctgccttttcaccgaggcactgccaccaccagctctagcagggatgctcctgagcttggcggacatacttagatcctaacgtgccagtgagacctggctgtggagagtagcactggcagccctgcctggactccacttggcatgataccagctccagaagggaagggagtggagcaggcagtgaggagagagcctgggggtcggctggggacagccgtatgtgctaggtaggagtggagggagatatgtttaccaaatgcctgtcctgccatcctcccaggtagtcagagtgagctacatcctgccccgccttcatttccatggaaacatggcagctaggacacggggtacaacagcagccaaattcttccccacctcccttacttcgaaaaaaagtttggaaccctggtccctatactctgcagtcagaagtgggactgagccatacatgcccttgaattcctccctgtctggccctccctctccagcaagcagggttttctttaacttggcagtgtgcagaggagaagtggtaacacccccaccccattcccctgcatcggagctcagtattcctacagggtaagaggtaggaatcttgctgggacgaggggagccagaagtggcaataaaagcgtgttgacctgggctacgcgggactccatgaattttccattctggcaactggaaggggaggtgtagaagggccctgcagaggacggcactggagactctcctaaacgccacacacctcccccttgctgcccgccacacgcgcgtctccttcaaccgatgcattgccaccctcccacagcctcgccctctgctctaaattaggtcgaaagagtcccagttccaatctaggaacagcataattttgttccaaaacctgatctgtgtttttctaattaaaattaatttgtcacccaaaacaggagggggatggggaggagggacatgagcactccgctggggccgggacggggaatgaggaggaagaaggccctctcttcccctacccgggccctccaggaccttctccctgagtcggctgtagggaggactccgaggaagctcggcgcggtgcgctgggcctcctggcgccgggaggagcgcgcgaggcaggcggggcgggcgcgtccggggcggggcgctggacttcgtgggtggtccccgccc

SEQ ID NO: 8 - DNA, 5420 bp, Murine
ggcggcctaggcccgcgcctgcgtcccccaacccggaggagatggggggctgtgctagctgcttgcccagcgagtgttcccggagtctcctcgccgcaccccgctatcgggcgctcagaccaggggctcggcctggcgggaaatagcctcgaccgaactgctgcggggacaaagagccgcgagttcgctccctgcccccagtgtctcctaagcgagccttctgctcctaggaaggcgcctttccagcagccgctgggaccctggccagtgtttagctcccaactagtaacagacggaaagaaaaaataaaattatccagaatctagaataaaaagtaagggccttttattctgggagaggacagttaaaaagcgcgctttcttgctaacttgcctttcttggttagtgccagagacgtgcactttataaacttatttaagtgacacggacccagaagtcgatgtcttgggttagaggttggagtgatgcgccctctgggttcagcggctttgaggagctcaccctgcagcgggctccgtttggggccccttggaggaagtaagaccaggaaccgtgatcctcagaatgattattcaaatctggattttcaaaactaagaactacactttcaccatcgcctctactgtctctcagtatctagcctcctgggtctggactaaaaggctgggaacttctgcccatgccccagactttataacgcacagagcaaatgttcagtatagtgcagttcagggttcctagctagattgcaaattgccaatgtctctgattctcagtctgtagctttctttctcttcggcctgatgctggggaagaaaatacgcgaagttttatatttcatgtttgctccttgaatttctacctatttctagtaaccctcctcccaaacacacactgttaccagacacacactgttaccgggagtccttcccaagagagggttcttggatctgacacaagaaagaattcagggcaagtccataaagtgaaagcaagtttattagaaaagtaaaggaataaagaatggctactccatagacagagcagccccaagggctgctggttgcccattgttagggttatttcttgatgatatgctaaacaaggggtggattattcatgcctcacctttttagaccatgtaaggtaacttcctgacgttgccatggcatttgtaaactgtcgtggtgctgatgggagcgtagcagtgaggacgaccagaggtcactctcgtcaacattttggttgtggtgggttttggccggcttctttaccgcaatctgttttatcagcaaggtatttctgacctgtatcttgtgccgacctcctatctcatcctgtgacttagaatgccttaatctgctgggaatgcagcccagtaggtctcagccttattttacccagcgcctattgaagatggagttgctctggttcaaatgcttctgacaccactactttagaatagctaatcaaatgtaggccgcaaaggtggttgaatgaaaggagattcttggggacagcagaaatcaggcttggtacacagaggtggagagaaaggcctgaaggatggtgaaaacctgccagggtggcatgccaagaaaggcaccactaagttctgcgggccttaagccctgcagttaaacaaagacttgtgcccagaagagcaaattttgcagaatgttcagtgaaattctgatttcctcctatcaggtttttattgctactgttacaaattaccagacatttagtagtttaaagcaacacaaatttatcttacattctggaagtaagaagtccaaaatgggtctcactgagctaagatcaaggtgtgagcagggctgtattccttcttgggggcttcaggggagaatccatctttcctgctcccagaggccgcgtgcatacctttgggtggtccctttctccaacttctgtcatcacatctcctttttctctgactctcctactgccccctgacaggacccctgtgattgtattggcccatctagattatccaggatgcccttcccccatgaagatcatgaatctcgccacgtctacaaagtcccttttagtatgtaaggaaagagatgcatcggttctagggattagggcatggacatttttggggtggcatgatttagctcattacatccccctttccagcctcatgaatgccttcaggtgcggggcatttgggaatgagctgatgttggatgctggcaagagtttgaggtaggtgttctctggtcttctttctgataattatgtaacgtctatcaagtgcttatgatgcaccaggcacatatttattatgaagttcaatacgcagccttggctattttctgacttatcctcatcctgaagaccagatatgttaacagggccagagaccaggttcaaaaaggagagcaggccaggtgcagtggctcatacctgtaatcccggcactttgggaggccaaggcaggaggatcacctgaggtcaggagtttgagaccagcctgggcaacagagggagaccctgtctgtacaaaacataaaaattagccaggcatggtgacatgttggcctctactgtctcttgctatctagcctcctgggtcagtcccagctactttggaagctaaggtggggaggatcacttgagcctgggggtgtgtaggggtgggggtgggaggcagggaggtgggtgtcgaggctgcagagacctgtgatcaccccactgcactccagtgagcaacagagaagactctttctctctctctctctctctctcacacacacacacacacacacacacacacacacacaaagcaagcagcacagcttcttccatcagcagttcttctgttctgttttgtaaacacccccaccccacccccaaaaataagagcaattagatatttcaaatcatcataggtaaagcacttaggggagggaataaagtaagggataaccacggtctagaactccagttggaaattaagcctccagagtcttctcattaccatatcgggaatgtggaaaatcaatcaaatgaatggaaatagcaaagggattccagaaatattttggacatcggtgctcactatataataaacaagaagcaatgcaatgttgagaggaagctgtaatcataattactaataaatgctattaggcgaattattacagtgtggaaaataagtaacagattcctacctcacactgtgcactgcaaagcattccagaagggtaaaagagctaagattttaaagtaaaacaaacaagaaaaccagaatatcatactgtcctagctctaggaagctacagtagacagcttttcagtgtatcttcccaacttgcaatgattccttcttctttggaaactggcatccacccctcaagccataggggtgagtcttgaggagccatatttatcctctaaggcctggtcccctaggcatagctgattggatccagctcaggcactctattcgtttcctagggctcccatcacaaagtaccacaaaaatagatcgcttaaaacaacagataattattgtttcacagttttggaggccggaagtctgaaatcaaggtgttgaccaggccatgctccccggaagcctccaggggaggatccttccttgtcctttccagcttctggtagctgggtattccttggcttgcggcagtataaatgcagactctgcctccctcttcatcttccctctgtgtctgtgtcccaattttcctcttctcataaggatgccagtcacattggatcaaggcccactgtactccagtatcctcatcttttttttttttttttttttttgagacaaagtcttgctctgtcacccaggctggagtgtagtgtcacgatcttggctcactgcaacctccacctcctgggttcaagtgattctcctgcctcagtctccctagtagctgggattacaggcacgcacttccacgccctgctaatttttgtatttttagtagagtcagggtttcaccatgttggtcaggatggtcttgaacttctgacctcaggtgatctgaccatctcagcctcccaaaatgctgggattacaagcgtgagccaccacgcgcctggcctccagtatcatcatcttaattaattacttctgcaatgaccctatctccaaataaggtcccaatctgaggtgcaggggtctagggcttcaacatatcttttttaagaagtcaaaattcaacccacaacaggcacctaacccaaacagtaacaaacagagttatttccctgggacttgggaattgagaatgaggttctagtcacagcctgggctggtctcatgaagaaagagaaggcaaagggaggctgaggtaccagccctcttatgctccagggactgattatggagaaagtctattcccagaaacaagagaaggaaggagtggagtgcggtgaggaggaagaagaggagtcctcctagaagcgcctttcctctataatcccagaacctttctgcagcggcactgctttccagccgtggcttccctatctgcttacagtaaagcctgttttgtttgcttagggaagctcaactagtttctcttgctcgcaagcagagttaatagaaaaactagaatcagagtaagatgctgtattagtactcttttgctgctgtaacaactttccacaaactcagcgctcaaaacaacacaaatttggccgggtgcggtggctcatgcctgtaatcctagcactttgggaggccaaggtgggtggatcacctaaggtcaggagtttgagaccagcctggccaacatggcgaaaccctgtctctactaaaagtataaaagttagctaggtgtggtggcacatgtctgtaatcccagctacttgggaggctgagacagaatcacttgaacgcaggaggcagaggttgcagtgagccaagatcatgccactacactccagcctggccgacagagtgagactgcatctcaaaaaaaaaaaaaaaaaaaaaaagcaacacaaattttttatcttacgttctgttggtcagaagtgtgctatgagtctcactgggttaaaaccaaggcatcagcagggttgcattccttgctggagattccagggacaatacatttctttgtcttttcctccttcttgaggctacctgctttccttggttggcggccccatcctccatcttccaagcctgcaagggcagggcaggtacttctcacactactgtctctctgcggccaggaaagattcttcactcctgtgattaggttgggcccatctgcataatctaagatactttccccatctcgaggaatgtaaccttaatcacaccatttgcaaagtcccttttgccatatgaggtaatatattcacaggtcctggggattaggata

SEQ ID NO: 9 - DNA, 5420 bp, Murine; identical to SEQ ID NO: 8

(19) United States
(12) Patent Application Publication
(10) Pub. No.: US 2009/0264354 A1
(43) Pub. Date: Oct. 22, 2009

(54) Penumbra Nucleic Acid Molecules, Proteins and Uses Thereof

(75) Inventors: Schickwann Tsai, Salt Lake City, UT (US); Zhong Chen, Sandy, UT (US)

Correspondence Address: THE MCCALLUM LAW FIRM, P. C., 685 BRIGGS STREET, PO BOX 929, ERIE, CO, 80516, US

(73) Assignee: University of Utah Research Foundation, Salt Lake City, UT (US)

(21) Appl. No.: 11/992,872

(22) PCT Filed: Sep. 28, 2006
(86) PCT No.: PCT/US06/38187

§ 371 Date: Mar. 28, 2008

Related U.S. Application Data
(60) Provisional application No. 60/721,842, filed on Sep. 28, 2005.

(51) Int. Cl. A61K 38/16; C07K 14/705; C12Q 1/68

(52) U.S. Cl. 514/12; 536/23.5; 435/6

(57) ABSTRACT

The present invention relates to murine and human Penumbra (for proerythroblast nu[new] Membrane) nucleic acid molecules, proteins and the uses thereof. The invention further relates to the use of Penumbra molecules for the detection of 7q31q32-related deletions, including such deletions associated with myeloid malignancies, particularly detection by hybridization using Penumbra-based probes.

### CROSS REFERENCE TO RELATED APPLICATIONS

This application claims priority from U.S. Provisional Patent Application No. 60/721,842 filed Sep. 28, 2005.

### FIELD OF THE INVENTION

The present invention relates to Penumbra gene nucleic acid molecules, proteins encoded by such nucleic acid molecules, antibodies raised against such proteins, and inhibitors of such proteins. The present invention also includes methods to obtain such proteins, nucleic acid molecules, antibodies, and inhibitory compounds. The present invention also includes methods of using such molecules in the detection of disease, including myeloid malignancies, such as acute myelogenous leukemias, myeloproliferative disorders, chronic myelogenous leukemia, juvenile myelomonocytic leukemia, transient myeloproliferative disorder and myelodysplastic syndromes. The present invention also includes methods of using such molecules to treat these same diseases.

### BACKGROUND OF THE INVENTION

Tetraspanins are integral membrane proteins characterized by the presence of four transmembrane domains. Tetraspanins are evolutionarily conserved, suggesting that they provide essential functions in multi-cellular organisms, including a role in signal transduction at the cell surface. A common theme emerging from studies of prototypical tetraspanins is that they function as organizers of supramolecular signaling complexes in cell membranes. The multiple interactions exhibited by many prototypical tetraspanins suggest involvement in diverse aspects of cellular physiology, including apoptosis and cell proliferative behavior.

### SUMMARY OF THE INVENTION

The present invention relates to a new member of the tetraspanin family, specifically murine and human Penumbra (for proerythroblast nu[new] membrane) nucleic acid molecules, proteins, and the uses thereof. In a particular embodiment, the present invention relates to isolated nucleic acid molecules having a nucleic acid sequence SEQ ID NO: 1, SEQ ID NO:2, SEQ ID NO:4, SEQ ID NO:6, SEQ ID NO:7, SEQ ID NO: 8, and/or SEQ ID NO:9, isolated nucleic acid molecules that encode proteins having amino acid sequences SEQ ID NO:3, and/or SEQ ID NO:5, and variants of such nucleic acid molecules.

The present invention also relates to recombinant molecules, recombinant viruses and recombinant cells that include a nucleic acid molecule of the present invention. The present invention also includes transgenic mice including a normal, mutated or otherwise disrupted (“knocked-out”) nucleic acid molecule of the present invention. Also included are methods to produce such nucleic acid molecules, recombinant molecules, recombinant viruses and recombinant cells. Also included are methods to produce a protein of the present invention.

The present invention also relates to methods of detecting 7q31q32-related deletions using Penumbra-based molecules. In a particular embodiment, the present invention relates to the detection of 7q31q32-related deletions in myeloid malignancies, particularly detection by hybridization using Penumbra-based probes.

The present invention also relates to methods of treating disease with Penumbra-based molecules, including myeloid malignancies, such as myelodysplastic syndromes, acute myelogenous leukemias and myeloproliferative disorders.

### DETAILED DESCRIPTION OF THE INVENTION

The present invention may be understood more readily by reference to the following detailed description of particular embodiments of the invention.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present compositions and/or methods are disclosed and described, it is to be understood that this invention is not limited to specific molecules or methods, as such may, of course, vary, unless it is otherwise indicated. It is also to be understood that the terminology used herein is for the purpose of describing particular embodiments only and is not intended to be limiting.

The present invention describes new genes involved in the regulation of hematopoiesis. In particular, the present invention is directed to mouse and human Penumbra (for proerythroblast nu[new] membrane) genes. Penumbra encodes a new member of the evolutionarily conserved tetraspanin membrane protein family that includes CD9, CD53, CD63, CD81, CD82, CD151 and peripherin. Penumbra is expressed in the cells of bone marrow and spleen and exhibits growth-suppressive activity in vitro.

The present invention provides for human and mouse Penumbra nucleic acid molecules, proteins encoded by such nucleic acid molecules, antibodies raised against such proteins, and inhibitors of such proteins. As used herein, Penumbra nucleic acid molecules and proteins encoded by such nucleic acid molecules are also referred to as Penumbra nucleic acid molecules and proteins of the present invention, respectively. Penumbra nucleic acid molecules and proteins of the present invention can be isolated from a individual or prepared recombinantly or synthetically. Penumbra nucleic acid molecules of the present invention can be RNA or DNA, or modified forms thereof, and can be double-stranded or single-stranded; examples of nucleic acid molecules include, but are not limited to, complementary DNA (cDNA) molecules, genomic DNA molecules, synthetic DNA molecules, DNA molecules which are specific tags for messenger RNA, and corresponding mRNA molecules. As such, a Penumbra nucleic acid molecule of the present invention is not intended to refer to an entire chromosome within which such a nucleic acid molecule is contained, however, a Penumbra nucleic acid molecule of the present invention may include all regions such as regulatory regions that control production of Penumbra proteins encoded by such a nucleic acid molecule (such as, but not limited to, transcription, translation or post-translation control regions) as well as the coding region itself, and any introns or non-translated coding regions. As used herein, the phrase “Penumbra protein” refers to a protein encoded by a Penumbra nucleic acid molecule.

The present invention also provides for biomarker DNA molecules that are specific tags for messenger RNA molecules. Such DNA molecules can correspond to an entire or partial sequence of a messenger RNA, and therefore, a DNA molecule corresponding to such a messenger RNA molecule (i.e. a cDNA molecule), can encode a full-length or partial-length protein. A nucleic acid molecule encoding a partial-length protein can be used directly as a probe or indirectly to generate primers to identify and/or isolate a cDNA nucleic acid molecule encoding a corresponding, or structurally related, full-length protein. A cDNA encoding a partial-length biomarker protein can also be used in a similar manner to identify a genomic nucleic acid molecule, such as a nucleic acid molecule that contains the complete gene including regulatory regions, exons and introns. Methods for using cDNA molecules and sequences encoding partial-length biomarker proteins to isolate nucleic acid molecules encoding full-length biomarker proteins and corresponding cDNA or genomic DNA molecules are well known in the art.

The proteins and nucleic acid molecules of the present invention can be obtained from their natural source, or can be produced using, for example, recombinant nucleic acid technology or chemical synthesis. Also included in the present invention is the use of these proteins and nucleic acid molecules as well as antibodies as disease detection tools or in the creation of transgenic animals, as well as in other applications.

It is to be noted that the term “a” or “an” entity refers to one or more of that entity; for example, a protein, a nucleic acid molecule and an antibody refers to “one or more” or “at least one” protein, nucleic acid molecule, antibody and therapeutic composition respectively. As such, the terms “a” (or “an”), “one or more” and “at least one” can be used interchangeably herein. It is also to be noted that the terms “comprising”, “including”, and “having” can be used interchangeably. According to the present invention, an isolated, or biologically pure, protein, is a protein that has been removed from its natural milieu. As such, “isolated” and “biologically pure” do not necessarily reflect the extent to which the protein has been purified. An isolated protein of the present invention can be obtained from its natural source, can be produced using recombinant DNA technology, or can be produced by chemical synthesis.

As used herein, isolated Penumbra proteins of the present invention can be full-length proteins or any homologue of such proteins. Examples of Penumbra homologue proteins include Penumbra proteins in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) such that the homologue includes at least one epitope capable of eliciting an immune response against a Penumbra protein, and/or of binding to an antibody directed against a Penumbra protein. That is, when the homologue is administered to an animal as an immunogen, using techniques known to those skilled in the art, the animal will produce an immune response against at least one epitope of a natural Penumbra protein. The ability of a protein to effect an immune response can be measured using techniques known to those skilled in the art.

As used herein, the term “epitope” refers to the smallest portion of a protein or other antigen capable of selectively binding to the antigen binding site of an antibody or a T cell receptor. It is well accepted by those skilled in the art that the minimal size of a protein epitope is about four to six amino acids. As is appreciated by those skilled in the art, an epitope can include amino acids that naturally are contiguous to each other as well as amino acids that, due to the tertiary structure of the natural protein, are in sufficiently close proximity to form an epitope. According to the present invention, an epitope includes a portion of a protein comprising at least 4 amino acids, at least 5 amino acids, at least 6 amino acids, at least 10 amino acids, at least 15 amino acids, at least 20 amino acids, at least 25 amino acids, at least 30 amino acids, at least 35 amino acids, at least 40 amino acids or at least 50 amino acids in length.

Nucleotide sequences are referred to by a sequence identifier number [SEQ ID NO]. The SEQ ID NOs correspond numerically to the sequence identifiers <400>1 [SEQ ID NO: 1], <400>2 [SEQ ID NO:2], etc. A summary of the sequence identifiers is provided in Table 1. A sequence listing is provided after the claims.

TABLE 1

| SEQ ID NO: | Description |
|---|---|
| 1 | The nucleic acid sequence of the mouse E6-3 probe |
| 2 | The cDNA sequence encoding a full-length mouse |
| | Penumbra protein |
| 3 | The amino acid sequence of the full-length mouse |
| | Penumbra protein |
| 4 | The cDNA sequence encoding a full-length human |
| | Penumbra protein |
| 5 | The amino acid sequence of the full-length human |
| | Penumbra protein |
| 6 | The nucleic acid sequence of the Pen-A FISH probe |
| 7 | The nucleic acid sequence of the Pen-B FISH probe |
| 8 | The nucleic acid sequence of the Pen-C FISH probe |
| 9 | The nucleic acid sequence of the Pen-D FISH probe |

In one embodiment of the present invention a Penumbra homologue protein exhibits an activity similar to its natural counterpart. Methods to detect and measure such activities vary by protein.

Penumbra homologue proteins can be the result of natural allelic variation or natural mutation. Penumbra protein homologues of the present invention can also be produced using techniques known in the art including, but not limited to, direct modifications to the protein or modifications to the gene encoding the protein using, for example, classic or recombinant DNA techniques to effect random or targeted mutagenesis.

Penumbra proteins of the present invention are encoded by biomarker nucleic acid molecules. As used herein, Penumbra nucleic acid molecules include nucleic acid sequences related to natural Penumbra genes. As used herein, Penumbra genes include all regions such as regulatory regions that control production of biomarker proteins encoded by such genes (such as, but not limited to, transcription, translation or post-translation control regions) as well as the coding region itself, and any introns or non-translated coding regions. As used herein, a nucleic acid molecule that “includes” or “comprises” a sequence may include that sequence in one contiguous array, or may include the sequence as fragmented exons such as is often found for a patient gene. As used herein, the term “coding region” refers to a continuous linear array of nucleotides that translates into a protein. A full-length coding region is that coding region that is translated into a full-length, i.e., a complete protein as would be initially translated in its natural milieu, prior to any post-translational modifications.

In one embodiment of the present invention, isolated Penumbra proteins are encoded by nucleic acid molecules that hybridize under stringent hybridization conditions to genes or other nucleic acid molecules encoding Penumbra proteins, respectively. The minimal size of such Penumbra proteins of the present invention is a size sufficient to be encoded by a nucleic acid molecule capable of forming a stable hybrid (i.e., hybridizing under stringent hybridization conditions) with the complementary sequence of a nucleic acid molecule encoding the corresponding natural protein. The size of a nucleic acid molecule encoding such a protein is dependent on the nucleic acid composition and the percent homology between the Penumbra nucleic acid molecule and the complementary nucleic acid sequence. It can easily be understood that the extent of homology required to form a stable hybrid under stringent conditions can vary depending on whether the homologous sequences are interspersed throughout a given nucleic acid molecule or are clustered (i.e., localized) in distinct regions on a given nucleic acid molecule.

The minimal size of a nucleic acid molecule capable of forming a stable hybrid with a gene encoding a Penumbra protein of the present invention is at least about 12 to about 15 nucleotides in length if the nucleic acid molecule is GC-rich and at least about 15 to about 17 bases in length if it is AT-rich. The minimal size of a nucleic acid molecule used to encode a Penumbra protein homologue of the present invention is from about 12 to about 18 nucleotides in length. Thus, the minimal size of Penumbra protein homologues of the present invention is from about 4 to about 6 amino acids in length. There is no limit, other than a practical limit, on the maximal size of a nucleic acid molecule encoding a Penumbra protein of the present invention because a nucleic acid molecule of the present invention can include a portion of a gene or cDNA or RNA, an entire gene or cDNA or RNA, or multiple genes or cDNA or RNA. In a particular embodiment, the size of a protein encoded by a nucleic acid molecule of the present invention depends on whether a full-length, fusion, multivalent, or functional portion of such a protein is desired.

Stringent hybridization conditions are determined based on defined physical properties of the Penumbra nucleic acid molecule to which the nucleic acid molecule is being hybridized, and can be defined mathematically. Stringent hybridization conditions are those experimental parameters that allow an individual skilled in the art to identify significant similarities between heterologous nucleic acid molecules. These conditions are well known to those skilled in the art. See, for example, Sambrook, et al., 1989, Molecular Cloning: A Laboratory Manual, Cold Spring Harbor Labs Press, and Meinkoth, et al., 1984, Anal. Biochem. 138, 267-284, each of which is incorporated by reference herein in its entirety. As explained in detail in the cited references, the determination of hybridization conditions involves the manipulation of a set of variables including the ionic strength (M, in moles/liter), the hybridization temperature (° C.), the concentration of nucleic acid helix destabilizing agents (such as formamide), the average length of the shortest hybrid duplex (n), and the percent G+C composition of the fragment to which an unknown nucleic acid molecule is being hybridized. For nucleic acid molecules of at least about 150 nucleotides, these variables are inserted into a standard mathematical formula to calculate the melting temperature, or Tₘ, of a given nucleic acid molecule. As defined in the formula below, Tₘ is the temperature at which two complementary nucleic acid molecule strands will disassociate, assuming 100% complementarity between the two strands:

Tₘ=81.5° C.+16.6 log M+0.41(% G+C)−500/n−0.61(% formamide).

For nucleic acid molecules smaller than about 50 nucleotides, hybrid stability is defined by the dissociation temperature (T_{d}), which is defined as the temperature at which 50% of the duplexes dissociate. For these smaller molecules, the stability at a standard ionic strength is defined by the following equation:

T_{d}=4(G+C)+2(A+T).

A temperature of 5° C. below T_{d} is used to detect hybridization between perfectly matched molecules.

Also well known to those skilled in the art is how base pair mismatch, i.e. differences between two nucleic acid molecules being compared, including non-complementarity of bases at a given location, and gaps due to insertion or deletion of one or more bases at a given location on either of the nucleic acid molecules being compared, will affect Tₘ or T_{d} for nucleic acid molecules of different sizes. For example, Tₘ decreases about 1° C. for each 1% of mismatched base pairs for hybrids greater than about 150 bp, and T_{d} decreases about 5° C. for each mismatched base pair for hybrids below about 50 bp. Conditions for hybrids between about 50 and about 150 base pairs can be determined empirically and without undue experimentation using standard laboratory procedures well known to those skilled in the art. These simple procedures allow one skilled in the art to set the hybridization conditions (by altering, for example, the salt concentration, the formamide concentration or the temperature) so that only nucleic acid hybrids with greater than a specified % base pair mismatch will hybridize. Because one skilled in the art can easily determine whether a given nucleic acid molecule to be tested is less than or greater than about 50 nucleotides, and can therefore choose the appropriate formula for determining hybridization conditions, he or she can determine whether the nucleic acid molecule will hybridize with a given gene under conditions designed to allow a desired amount of base pair mismatch.

Hybridization reactions are often carried out by attaching the nucleic acid molecule to be hybridized to a solid support such as a membrane, and then hybridizing with a labeled nucleic acid molecule, typically referred to as a probe, suspended in a hybridization solution. Examples of common hybridization reaction techniques include, but are not limited to, the well-known southern and northern blotting procedures. Typically, the actual hybridization reaction is done under non-stringent conditions, i.e., at a lower temperature and/or a higher salt concentration, and then high stringency is achieved by washing the membrane in a solution with a higher temperature and/or lower salt concentration in order to achieve the desired stringency.

For example, if the skilled artisan wished to identify a nucleic acid molecule that hybridizes under conditions that would allow less than or equal to 30% pair mismatch with a Penumbra nucleic acid molecule of the present invention of about 150 bp in length or greater, the following conditions could be used. Assume for example that the average G+C content of patient DNA is about 51%, as calculated from known patient nucleic acid sequences. The unknown nucleic acid molecules would be attached to a support membrane, and the 150 bp probe would be labeled, e.g. with a radioactive tag. The hybridization reaction could be carried out in a solution comprising 2×SSC in the absence of nucleic acid helix destabilizing compounds, at a temperature of about 37° C. (low stringency conditions). Solutions of differing concentrations of SSC can be made by one of skill in the art by diluting a stock solution of 20×SSC (175.3 gram NaCI and about 88.2 gram sodium citrate in 1 liter of water, pH 7) to obtain the desired concentration of SSC. The skilled artisan would calculate the washing conditions required to allow up to 20% base pair mismatch. For example, in a wash solution comprising 1×SSC in the absence of nucleic acid helix destabilizing compounds, the Tₘ of perfect hybrids would be about 85.4° C.:

81.5° C.+16.6 log(0.15M)+(0.41×51)−(500/150)−(0.61×0)=85.4° C.

Thus, to achieve hybridization with nucleic acid molecules having about 20% base pair mismatch, hybridization washes would be carried out at a temperature of less than or equal to 65.4° C. It is thus within the skill of one in the art to calculate additional hybridization temperatures based on the desired percentage base pair mismatch, formulae and G/C content disclosed herein. For example, it is appreciated by one skilled in the art that as the nucleic acid molecule to be tested for hybridization against nucleic acid molecules of the present invention having sequences specified herein becomes longer than 150 nucleotides, the Tₘ for a hybridization reaction allowing up to 20% base pair mismatch will not vary significantly from 65.4° C. Similarly, to achieve hybridization with nucleic acid molecules having about 10% base pair mismatch, hybridization washes would be carried out at a temperature of less than or equal to 75.4° C. and to achieve hybridization with nucleic acid molecules having about 5% base pair mismatch, hybridization washes would be carried out at a temperature of less than or equal to 80.4° C.

Furthermore, it is known in the art that there are commercially available computer programs for determining the degree of similarity between two nucleic acid or protein sequences. These computer programs include various known methods to determine the percentage identity and the number and length of gaps between hybrid nucleic acid molecules or proteins. Particular methods to determine the percent identity among amino acid sequences and also among nucleic acid sequences include analysis using one or more of the commercially available computer programs designed to compare and analyze nucleic acid or amino acid sequences. These computer programs include, but are not limited to, the SeqLab® Wisconsin Package™ Version 10.0-UNIX sequence analysis software, available from Genetics Computer Group, Madison, Wis. (hereinafter “SeqLab”; and DNAsis® sequence analysis software, version 2.0, available from Hitachi Software, San Bruno, Calif. (hereinafter “DNAsis”). Such software programs represent a collection of algorithms paired with a graphical user interface for using the algorithms. The DNAsis and SeqLab software, for example, employ a particular algorithm, the Needleman-Wunsch algorithm to perform pair-wise comparisons between two sequences to yield a percentage identity score, see Needleman, S. B. and Wunch, C. D., 1970, J. Mol. Biol., 48, 443, which is incorporated herein by reference in its entirety. Such algorithms, including the Needleman-Wunsch algorithm, are commonly used by those skilled in the nucleic acid and amino acid sequencing art to compare sequences. A particular method to determine percent identity among amino acid sequences and also among nucleic acid sequences includes using the Needleman-Wunsch algorithm, available in the SeqLab software, using the Pairwise Comparison/Gap function with the nwsgapdna.cmp scoring matrix, the gap creation penalty and the gap extension penalties set at default values, and the gap shift limits set at maximum (hereinafter referred to as “SeqLab default parameters”). An additional method to determine percent identity among amino acid sequences and also among nucleic acid sequences includes using the Higgins-Sharp algorithm, available in the DNAsis software (hereinafter “DNAsis”), with the gap penalty set at 5, the number of top diagonals set at 5, the fixed gap penalty set at 10, the k-tuple set at 2, the window size set at 5, and the floating gap penalty set at 10.

One embodiment of the present invention includes a Penumbra protein. A particular Penumbra protein includes a protein encoded by a nucleic acid molecule that hybridizes under conditions that allow less than or equal to 30% base pair mismatch, under conditions that allow less than or equal to 20% base pair mismatch, under conditions that allow less than or equal to 10% base pair mismatch, under conditions that allow less than or equal to 8% base pair mismatch, under conditions that allow less than or equal to 5% base pair mismatch or under conditions that allow less than or equal to 2% base pair mismatch with a nucleic acid molecule of the present invention.

Another Penumbra protein of the present invention includes a protein that is encoded by a nucleic acid molecule that is at least 70%, at least 80%, at least 90% identical, at least 92% identical, at least 95% identical or at least 98% identical to a nucleic acid molecule of the present invention; also included are fragments (i.e. portions) of such proteins encoded by nucleic acid molecules that are at least 50 nucleotides. Percent identity as used herein may be determined, for example, using the Needleman-Wunsch algorithm, available in the SeqLab software using default parameters.

Another embodiment of the present invention is an isolated nucleic acid molecule comprising a Penumbra nucleic acid molecule, i.e. a nucleic acid molecule that can be isolated from a patient cDNA library. The identifying characteristics of such nucleic acid molecules are heretofore described. A nucleic acid molecule of the present invention can include an isolated natural Penumbra gene or a homologue thereof, the latter of which is described in more detail below. A nucleic acid molecule of the present invention can include one or more regulatory regions, full-length or partial coding regions, or combinations thereof. The minimal size of a nucleic acid molecule of the present invention is a size sufficient to allow the formation of a stable hybrid (i.e., hybridization under stringent hybridization conditions) with the complementary sequence of another nucleic acid molecule. As such, the minimal size of a biomarker nucleic acid molecule of the present invention is from 12 to 18 nucleotides in length.

In accordance with the present invention, an isolated nucleic acid molecule is a nucleic acid molecule that has been removed from its natural milieu (i.e., that has been subjected to human manipulation) and can include DNA, RNA, or derivatives of either DNA or RNA. As such, “isolated” does not reflect the extent to which the nucleic acid molecule has been purified. Isolated biomarker nucleic acid molecules of the present invention, or homologues thereof, can be isolated from a natural source or produced using recombinant DNA technology (e.g., polymerase chain reaction (PCR) amplification or cloning) or chemical synthesis. Isolated Penumbra nucleic acid molecules, and homologues thereof, can include, for example, natural allelic variants and nucleic acid molecules modified by nucleotide insertions, deletions, substitutions, and/or inversions in a manner such that the modifications do not substantially interfere with the nucleic acid molecule's ability to encode a Penumbra protein of the present invention.

A Penumbra nucleic acid molecule homologue of the present invention can be produced using a number of methods known to those skilled in the art, see, for example, Sambrook et al., ibid., which is incorporated by reference herein in its entirety. For example, nucleic acid molecules can be modified using a variety of techniques including, but not limited to, classic mutagenesis and recombinant DNA techniques such as site-directed mutagenesis, chemical treatment, restriction enzyme cleavage, ligation of nucleic acid fragments, PCR amplification, synthesis of oligonucleotide mixtures and ligation of mixture groups to “build” a mixture of nucleic acid molecules, and combinations thereof. Nucleic acid molecule homologues can be selected by hybridization with biomarker nucleic acid molecules or by screening the function of a protein encoded by the nucleic acid molecule (e.g., ability to elicit an immune response against at least one epitope of a Penumbra protein or to effect biomarker protein activity).

An isolated Penumbra nucleic acid molecule of the present invention can include a nucleic acid sequence that encodes at least one Penumbra protein of the present invention respectively, examples of such proteins being disclosed herein. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a Penumbra protein.

Knowing the nucleic acid sequences of certain Penumbra nucleic acid molecules of the present invention allows one skilled in the art to, for example, (a) make copies of those nucleic acid molecules, (b) obtain nucleic acid molecules including at least a portion of such nucleic acid molecules (e.g., nucleic acid molecules including full-length genes, full-length coding regions, regulatory control sequences, truncated coding regions), (c) obtain other Penumbra nucleic acid molecules and (d) mutate such nucleic acid molecules. Such nucleic acid molecules can be obtained in a variety of ways including screening appropriate expression libraries with antibodies of the present invention; traditional cloning techniques using oligonucleotide probes of the present invention to screen appropriate libraries; and PCR amplification of appropriate libraries or DNA using oligonucleotide primers of the present invention. Libraries to screen or from which to amplify nucleic acid molecules include cDNA libraries as well as genomic DNA libraries. Similarly, DNA sources to screen or from which to amplify nucleic acid molecules include cDNA and genomic DNA. Techniques to clone and amplify genes are disclosed, for example, in Sambrook et al., ibid.

Site-specific mutagenesis of the nucleic acid molecules of the present invention can be a useful technique in the preparation of individual peptides, or biologically functional equivalent proteins or peptides, through specific mutagenesis of the underlying nucleic acid sequence. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is used, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

One embodiment of the present invention includes a recombinant vector, which includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that are derived from a species other than the species from which the nucleic acid molecule(s) are derived. The vector can be either RNA or DNA, either prokaryotic or eukaryotic, and typically is a virus or a plasmid. Recombinant vectors can be used in the cloning, sequencing, and/or otherwise manipulating of Penumbra nucleic acid molecules of the present invention.

One type of recombinant vector, referred to herein as a recombinant molecule, comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. In a particular embodiment, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Expression vectors of the present invention can direct gene expression in bacterial, yeast, insect and mammalian cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences that control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Transcription control sequences of the present invention include those that function in bacterial, yeast, or insect and mammalian cells, such as, but not limited to, tac, lac, trp, trc, oxy-pro, omp/lpp, rrnB, bacteriophage lambda (such as lambda p_{L} and lambda p_{R} and fusions that include such promoters), bacteriophage T7, T7lac, bacteriophage T3, bacteriophage SP6, bacteriophage SP01, metallothionein, alpha-mating factor, Pichia alcohol oxidase, alphavirus subgenomic promoter, antibiotic resistance gene, baculovirus, Heliothis zea insect virus, vaccinia virus, herpesvirus, raccoon poxvirus, other poxvirus, adenovirus, cytomegalovirus (such as immediate early promoter), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with patients, such as human transcription control sequences. Suitable nucleic acid molecules to include in recombinant vectors of the present invention are as disclosed herein.

Recombinant molecules of the present invention may also (a) contain secretory signals (i.e., signal segment nucleic acid sequences) to enable an expressed Penumbra protein of the present invention to be secreted from the cell that produces the protein and/or (b) contain fusion sequences which lead to the expression of nucleic acid molecules of the present invention as fusion proteins. Examples of suitable signal segments include any signal segment capable of directing the secretion of a protein of the present invention. Signal segments include, but are not limited to, tissue plasminogen activator (t-PA), interferon, interleukin, growth hormone, histocompatibility and viral envelope glycoprotein signal segments. Suitable fusion segments encoded by fusion segment nucleic acids are disclosed herein. In addition, a nucleic acid molecule of the present invention can be joined to a fusion segment that directs the encoded protein to the proteosome, such as a ubiquitin fusion segment. Eukaryotic recombinant molecules may also include intervening and/or untranslated sequences surrounding and/or within the nucleic acid sequences of nucleic acid molecules of the present invention.

Another embodiment of the present invention includes a recombinant cell comprising a host cell transformed with one or more recombinant molecules of the present invention, Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. It is to be noted that a cell line refers to any recombinant cell of the present invention that is not a transgenic animal.

Transformed nucleic acid molecules of the present invention can remain extrachromosomal or can integrate into one or more sites within a chromosome of the transformed (i.e., recombinant) cell in such a manner that their ability to be expressed is retained. Nucleic acid molecules with which to transform a cell include Penumbra nucleic acid molecules disclosed herein.

Suitable host cells to transform include any cell that can be transformed with a nucleic acid molecule of the present invention. Host cells can be either untransformed cells or cells that are already transformed with at least one nucleic acid molecule (e.g., nucleic acid molecules encoding one or more proteins of the present invention). Host cells of the present invention either can be enpatientenously (i.e., naturally) capable of producing Penumbra proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite (including helminth, protozoa and ectoparasite), other insect, other animal and plant cells. Host cells of the present invention include all cell types, including bacteria, mycobacterial, yeast, insect and mammalian cells. In a particular embodiment, the host cell is a mouse cell. Methods of the present invention are applicable to all cell types and organisms.

Such transformation may produce a therapeutic benefit to the host or it may merely be useful to have it produced in the host for any reason. Transformation of a host cell with a Penumbra product may function to increase gene expression and replace a gene product not produced in the endogenous host cell and such deficient product may cause disease. The increase in gene expression may only be successful in a minimal number of cells but such increase may contribute to better health for the host suffering from disease.

In a particular embodiment, a recombinant cell is produced by transforming a host cell with one or more recombinant molecules, each comprising one or more nucleic acid molecules of the present invention operatively linked to an expression vector containing one or more transcription control sequences, examples of which are disclosed herein. The phrase operatively linked refers to insertion of a nucleic acid molecule into an expression vector in a manner such that the molecule is able to be expressed when transformed into a host cell.

A recombinant cell of the present invention includes any cell transformed with at least one of any nucleic acid molecule of the present invention. Suitable nucleic acid molecules as well as suitable recombinant molecules with which to transfer cells are disclosed herein. Recombinant cells of the present invention can also be co-transformed with one or more recombinant molecules including Penumbra nucleic acid molecules encoding one or more proteins of the present invention and one or more other nucleic acid molecules encoding other compounds.

The present invention can be utilized in both somatic and germ line cells to effect protein expression of any Penumbra protein.

The recombinant cells of the present invention can be used for any purpose. In a particular embodiment, such use is for research, development, diagnostic or therapeutic purpose.

The Penumbra molecules of the present invention may be used to create transgenic animals. They may further be used to create novel experimental systems, such as animal models, cell-based assays, in-vitro assays, and the like. Additionally, they may be used to create animals or cells for the production of antibodies, vaccines and the like. They may further be useful to create components of novel regulatable expression systems for use in animal or cell culture systems synthetic or chimeric transcriptional regulators or other regulatory proteins, cells for autologous or heterologous transplantation, and the like. They may also be useful in generating cell lines for in-vitro ADME/Tox applications or high-throughput screening. Marked or tagged cells or tissues may also be created for experimental, diagnostic or therapeutic purposes.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

Isolated Penumbra proteins of the present invention can be produced in a variety of ways, including production and recovery of natural proteins, production and recovery of recombinant proteins, and chemical synthesis of the proteins. In one embodiment, an isolated protein of the present invention is produced by culturing a cell capable of expressing the protein under conditions effective to produce the protein, and recovering the protein. In a particular embodiment, the cell to culture is a recombinant cell of the present invention. Effective culture conditions include, but are not limited to, effective media, bioreactor, temperature, pH and oxygen conditions that permit protein production. An effective medium refers to any medium in which a cell is cultured to produce a Penumbra protein of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the protein”, as well as similar phrases, refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. In a particular embodiment, proteins of the present invention are retrieved in “substantially pure” form. As used herein, “substantially pure” refers to a purity that allows for the effective use of the protein as a therapeutic composition or diagnostic.

The present invention also includes isolated (i.e., removed from their natural milieu) antibodies that selectively bind to a protein of the present invention. As used herein, the term “selectively binds to” a protein refers to the ability of antibodies of the present invention to preferentially bind to specified proteins of the present invention. Binding can be measured using a variety of methods standard in the art including enzyme immunoassays (e.g., ELISA), immunoblot assays, etc.; see, for example, Sambrook et al., ibid., and Harlow, et al., 1988, Antibodies, a Laboratory Manual, Cold Spring Harbor Labs Press; Harlow et al., ibid., is incorporated by reference herein in its entirety. A particular antibody of the present invention selectively binds to a Penumbra protein in such a way as to inhibit the function of that protein.

Isolated antibodies of the present invention can include antibodies in serum, or antibodies that have been purified to varying degrees. Antibodies of the present invention can be polyclonal or monoclonal, or can be functional equivalents such as antibody fragments and genetically-engineered antibodies, including single chain antibodies or chimeric antibodies that can bind to one or more epitopes.

The present invention also relates to methods for the diagnosis of disease in patients. In one embodiment, a biological sample from a patient to be diagnosed is analyzed using fluorescent in situ hybridization (FISH) using a nucleic acid probe designed from human Penumbra nucleic acid sequence. The Penumbra FISH probe may be used as a marker for clinical FISH analysis of the chromosome 7q31-32 region for the detection of 7q31q32-related deletions in myeloid malignancies. In a particular embodiment, such FISH probe is selected from the group consisting of SEQ ID No. 6, 7, 8, and 9.

Diseases or indications capable of being detected by the present invention include myeloid malignancies. Such disease include, but are not limited to, myelodysplastic syndromes, acute myelogenous leukemias, myeloproliferative disorders, chronic myelogenous leukemia, juvenile myelomonocytic leukemia, transient myeloproliferative disorder and myelodysplastic syndromes.

In a particular embodiment, the methods of the present invention may be used to detect minimal residual disease, i.e. the presence of disease that was not eliminated through treatment, in order to measure the effectiveness of treatment. The methods of the present invention may also be used for determining the stage of disease.

In a particular embodiment, the molecules of the present invention are further isolated and the isolated molecules are used for the detection of disease using other diagnostic formats known to those of skill in the art. A suitable format includes binding assays, including assays that utilize antibodies raised against biological molecules of the present invention, including an ELISA format, or formatting the assay into a kit, such as a lateral flow or flow-through diagnostic format, or utilizing a biochip.

The present invention also relates to methods of treating myeloid malignancies with Penumbra-based molecules, such as myelodysplastic syndromes, acute myelogenous leukemias, myeloproliferative disorders, chronic myelogenous leukemia, juvenile myelomonocytic leukemia, transient myeloproliferative disorder and myelodysplastic syndromes.

Penumbra-based molecules of the present invention may be made and/or isolated by any technique known in the art.

Penumbra-based molecules of the present invention may be administered in any therapeutically effective manner. In addition to the formulations for parenteral administration, such as intravenous or intramuscular injection, other alternative methods of administration of the present invention may also be used, including but not limited to intradermal administration, pulmonary administration, buccal administration, transdermal and transmucosal administration. Transmucosal administration may include, but is not limited to, ophthalmic, vaginal, rectal and intranasal. All such methods of administration are well known in the art.

In a particular embodiment, the Penumbra-based molecules of the present invention may be administered intranasally, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5.

Antimicrobial preservatives, similar to those used in ophthalmic preparations, and appropriate drug stabilizers, if required, may be included in any of the formulations. Preservatives and other additives may be selected from the group consisting of, but not limited to, antimicrobials, anti-oxidants, chelating agents, inert gases and the like. Various commercial nasal preparations are known and include, for example, antibiotics and antihistamines and are used for asthma prophylaxis.

In another embodiment, Penumbra-based molecules of the present invention are applied topically. Such controlled release compositions include, but are not limited to, lotions, ointments, creams, gels, drops, suppositories, sprays, liquids and powders. Conventional pharmaceutical carriers, aqueous, powder or oily bases, thickeners and the like may be necessary or desirable.

Penumbra-based molecules of the present invention can be formulated in any excipient the biological system or entity can tolerate. Examples of such excipients include water, saline, Ringers solution, dextrose solution, Hank's solution and other aqueous physiologically balanced salt solutions. Nonaqueous vehicles, such as fixed oils, polyethylene glycol and injectable organic esters such as ethyl oleate may also be used. Other useful formulations include suspensions containing viscosity-enhancing agents, such as sodium carboxymethylcellulose, sorbitol or dextran.

Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

Pharmaceutical carriers for Penumbra-based molecules of the present invention are known to those skilled in the art. Those most typically utilized are likely to be standard carriers for administration to humans including solutions such as sterile water, saline and buffered solutions at physiological pH.

The Penumbra-based molecules of the present invention may be suspended in any aqueous solution or other diluent for injection in a human or animal patient in need of treatment. Aqueous diluent solutions may further include a viscosity enhancer selected from the group consisting of sodium carboxymethylcellulose, sucrose, mannitol, dextrose, trehalose and other biocompatible viscosity enhancing agents. The viscosity may be adjusted to a value between 2 centipoise (cp) and 100 cp, preferably between 4 and 40 cp.

In a particular embodiment, a surfactant may be included in the diluent to enhance suspendability of the Penumbra-based molecules. Surfactants may be selected from the group consisting of, but not limited to, polysorbates and other biocompatible surfactants. Surfactants are used at a concentration of between 0 and 5% (w/w), preferably between 0.1 and 1% w/w.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

This example describes the isolation and sequencing of mouse Penumbra nucleic acid sequence. Mouse multipotent hematopoietic cell line EML C1 (Tsai et al. 1993) was maintained in Iscove Modified Dulbecco's medium (IMDM: Gibco, Grand Island, N.Y.) plus 20% horse serum (HS: Gibco) and 8% (vol./vol.) BHK/MKL-conditioned medium (CM).

cDNA Representational Difference Analysis (RDA) was performed on EML C1 using a synergistic promyelocyte cell line, MPRO (Tsai and Collins, 1993), as the subtractor, resulting in an enrichment of genes expressed in the erythroid lineage, as follows. Double-stranded cDNAs were digested with DpnII and ligated with R-Bgl-12 and R-Bgl-24 linker/primers and amplified by polymerase chain reaction (PCR) to generate a representation of cDNAs. The MPRO representation (the “drive”) was subsequently digested with DpnII to remove linker/primers. The EML C1 cDNA representation (the “tester”) was digested with DpnII to remove the linker/primers, gel-purified, and religated with a second set of linker/primers (J-Bgl-12 and J-Bgl-24). The J-Bgl-12/24-ligated EML C1 cDNA representation was mixed with 100-fold excess of melted MPRO cDNA representation and hybridized at 67 C for 24 hours. Common sequences formed tester:driver duplexes that contained the linker/primer at only one end and could be amplified only in a linear fashion by PCR. The ratio of tester to driver DNAs were 1:8000 and 1:40,000 for the 2^{nd} and 3^{rd} rounds of RDA, respectively. The final products were gel purified and cloned in Bluescript SK(+) (Stratagene, La Jolla, Calif.) for sequencing.

One of the cDNA's identified, referred to as E6-3 (SEQ ID NO 1), was a 566 base pair molecule that was then radio-labeled and used as a probe to screen an oligo d(T)-primed cDNA library of EML C1. The screen yielded a 2017 nucleotide molecule SEQ ID NO:2, having an open reading frame of 852 nucleotides, spanning nucleotides 229-1080. This open reading frame encodes a protein of 283 amino acids (SEQ ID NO:3) with an apparent molecular weight of 26 kD.

### Example 2

This example describes the isolation and sequencing of human Penumbra nucleic acid sequence. Using murine Penumbra sequences described in Example 1 as a probe, the human Penumbra sequence was isolated from a normal human bone marrow cDNA library and a human BAC library. The resulting nucleic acid molecule was 1919 base pairs in length (SEQ ID NO:4).

Sequence analysis indicates that the human Penumbra (SEQ ID NO:4) encodes a full-length protein of 283 amino acids (SEQ ID NO:5) and is 97% identical to the murine Penumbra protein. This high degree of sequence homology suggests that Penumbra serves an important function. An analysis of an expressed sequence tag (est AI480218) corresponding to the end of the 3′ untranslated region of human Penumbra cDNA (SEQ ID NO:4) is predicted to be at chromosome 7q32.1 in the National Center for Biotechnology Information (NCBI) human genome database. This region of chromosome 7 is a hotspot for cytogenetic abnormalities in myeloid malignancies, including myelodysplastic syndromes (MDS), myeloproliferative disorders (MPD) and acute myeloid leukemias (AML).

### Example 3

This example describes the use of human Penumbra nucleic acid sequences in FISH analysis for the detection of 7q31q32-related deletions in myeloid malignancies. A DNA probe of the human Penumbra consisting of an equimolar mixture of four DNA fragments (“Pen-A-D”) (SEQ ID NO:6-9) that covered the entire coding region of Penumbra plus parts of its promoter and intron 9, was created by excising the four DNA fragments from BAC clone RP11286H14 (obtained from Children's Hospital Oakland Research Institute, Oakland, Calif.) by restriction enzyme digestion, gel-purifying, ligating the fragments into pBluescript SK(+) (Stratagene, La Jolla, Calif.) and transforming the ligated product into XL-1 Blue MRF′ E. Coli (Stratagene, La Jolla, Calif.). Probes Pen A-D have the following sequences: Pen A, having 2158 nucleotides is denoted SEQ ID NO:6, Pen B, having 1216 nucleotides is denoted as SEQ ID NO:7, Pen C, having 11,922 nucleotides is denoted as SEQ ID NO:8, and Pen D, having 5420 nucleotides is denoted as SEQ ID NO:9. The DNA sequences were verified by cycle sequencing. Pen-A-D were labeled by nick translation using 4 dNTPs and SpectrumOrange dUTP according to the manufacturer's instructions (Vysis, Downer's Grove, Ill.).

Bone marrow (BM) samples were collected from seven patients with myeloid malignancies at presentation as part of their diagnostic workup. The clinical diagnoses of these patients were based on the morphology, cytochemical staining and immunophenotyping of BM cells. Karyotyping was performed on BM cells that had been cultured for 24 hours without added cytokines. Chromosomes were characterized by the standard trypsin G-banding method and the karyotypes were described according to the International System for Human Cytogenetic Nomenclature (ISCN 1995).

Cell preparations for FISH were made on frozen cytogenetic cell suspensions and FISH was performed following the standard Vysis LSI protocol. Bone marrow samples from 20 individuals without hematological malignancies and with normal karyotypes were used as controls and 200 nuclei were evaluated for each specimen. Means and standard deviations (SD) of the percentages of nuclei with 1, 2, and 3 Penumbra hybridization signals were calculated. Results were considered abnormal if the percentage of nuclei with abnormal (single) hybridization signals was greater than 3 SD from the mean. 8.4% was established as the cut-off value for interpretation of the Penumbra deletion.

Among the seven cases of myeloid malignancies analyzed, five showed deletions of the Penumbra gene. The percentages of positively scored cells by FISH ranged from 9.2% to 66.5%. It is noteworthy that cases 6 and 7 without Penumbra gene deletions had chromosomal deletions at breakpoints distal to 7q32, whereas cases 1-5 with Penumbra gene deletions had cytogenetic deletions in 7q22q36 (cases 1-3), 7q31.2q36 (case 4) and 7q22q32 (case 5). Accordingly, these findings provide the foundation for using the Penumbra probe in FISH detection of 7q31q32-related deletions in myeloid malignancies.

### Example 4

This example describes the location of Penumbra in various hematopoietic tissues or cells by quantitative reverse-transcription-polymerase chain reaction (RT-PCR). A variety of mouse tissues were examined and Penumbra appeared to be highly expressed in bone marrow and spleen. Most of the expression in bone marrow appeared to be in the TER119⁺ fraction, which includes all erythroblasts. Little expression appeared to be located in nonerythroid tissues or cells such as thymus, Gr1⁺ neutrophils, CD3⁺ T cells, B220⁺ B cells, CD11c+monocytes or NK1.1⁺ natural killer cells. The expression in spleen may have been due to extramedullary hematopoiesis in this organ in mice. Northern analyses of poly(A)⁺ RNA detected lower levels of expression in liver, brain and kidney.

### Example 5

This example describes the apparent subcellular location of Penumbra. An expression vector, pcDNA3.1/Pen-Myc, was constructed to express murine Penumbra as a fusion protein with the 11-aa Myc and 6-aa His tags in C-terminus. Initially, the entire coding region of Penumbra (SEQ ID 2) excluding the stop codon was cloned in frame into pcDNA3.1 that contained sequences encoding Myc and His tags and a neo expression cassette. (Invitrogen, Carlsbad, Calif.) In vitro translation of pcDNA3.1/Pen-Myc in the presence of canine pancreatic microsomal membranes appeared to yield a 29-kD fusion protein. pcDNA3.1/Pen-Myc was then transfected into BaF3 and FLDS-19 cells, followed by staining with a rhodamine-conjugated, anti-Myc monoclonal antibody (MAb), 9E10 (Millipore, Billerica, Mass.). Most of the Penumbra-Myc fusion protein appeared to be found on the surface of cells or in the Golgi apparatus.

### Example 6

This example describes the analysis of the protein structure of Penumbra protein. NIH3T3 fibroblasts were co-transfected with pcDNA3.1/Pen-Myc and pEGFP NI/Pen. The latter expressed murine Penumbra (SEQ ID NO:3) as a fusion protein with enhanced green fluorescent protein (EGFP) in its C-terminus. The Pen-Myc fusion protein was first immunoprecipitated from cell lysates using the anti-Myc MAb, Western blotted and sequentially probed with anti-Myc and anti-EGFP antibodies. It appeared that both Pen-Myc/Pen-Myc and Pen-Myc/Pen-EGFP dimers were apparent in the immunoprecipitates under non-reducing conditions and they appeared to dissociate into monomers after reduction indicating they appear to form disulfide-bonded homodimers. It appeared that under reducing conditions, all Pen-Myc fusion proteins existed as 29-kD monomers. In contrast, under non-reducing conditions, it appeared that about half of Pen-Myc protein existed as 58-kD homodimers, while the remaining Pen-Myc protein appeared to exist either as 29-kDa monomers or 89-kDa dimers with Pen-EGFP. Treatment of the immunoprecipitates with iodoacetate to block free thiols prior to the addition of Western sample buffer did not appear to affect the appearance of homodimers. The same blot was then stripped and reprobed with anti-EGFP antibodies to reveal an apparent ˜60-kD Pen-EGFP monomer and the 89-kD Pen-Myc/Pen-EGFP dimer. This study indicates that nearly half of Pen-Myc protein exists as disulfide-bonded homodimers.

### Example 7

This example describes the construction of a knock-out mouse with a Penumbra deletion by homologous recombination. To avoid any pathology resulting from Neo, the targeting vector featured a self-excising Cre-Neo cassette flanked by two lox P sites (Bunting et al. 1999). Cre expression was then controlled by a testis-specific promoter. As the Penumbra^{+/-} Embryonic Stem (ES) cells passed through the testes of male chimeras, Cre was induced and mediated excision of the Cre-Neo cassette resulting in the deletion of part of TM₁, ECD₁, TM₂ and part of TM₃ of the Penumbra protein and resulted in the production of an aberrantly spliced mRNA. Genotyping was done by PCR on all mice.

### Example 8

This example describes the effect of Penumbra gene deletions in the knock-out mice created in Example 7. The knock-out mice appeared to be viable and fertile. When examined at 3 months of age, they had had similar numbers of red blood cells (RBC) (9.32±1.05 vs. 9.51±0.85×10⁶/μl in WT; n=40 and 33, respectively), hematocrit (46.77±3.92 vs. 47.11±3.72% in WT (stands for?)), white blood cells (WBC) (8.60±2.67 vs. 7.70±2.54×10³/μl in WT) and platelets (826.46±168.57 vs. 919.78±151.19×10³/μl in WT) as normal wild-type littermates. However, the blood smears of 30-40% of young knock-out mice appeared to contain RBC that were basophilic (or polychromatophilic) and larger. Some of the basophilic RBC appeared to have a “target cell” appearance, reflecting a decreased cytoplasm (hemoglobin)-to-cell surface ratio. Abnormal RBC may be referred to as “basophilic macrocytes” When examined at ˜1 year (range 6-17 months) of age, the knock-out mice as a group appeared to have significantly lower RBC numbers (7.71±0.88 vs. 8.88±0.51×10⁶/μl in WT; n=12 for both; p=0.004 by paired t-test) and hematocrits (38.95±5.52 vs. 46.21±3.62% in WT; p=0.003). Furthermore, about 40-60% of the 1-year-old knock-out mice appeared to have increased percentages of basophilic macrocytes in blood smears. In the more severe cases, 50% or more of the RBC appeared to be basophilic macrocytes. No consistent abnormalities were apparent in the white cells or platelets.

When mice with increased percentages (>4%) of basophilic macrocytes were examined as a group, they appeared to have even lower numbers of RBC (6.24±1.87 vs. 8.88±0.51×10⁶/μl in WT; n=9 and 12, respectively; p=0.0002 by unpaired t-test) and hematocrits (36.63±8.28 vs. 46.21±3.62% in WT; p=0.002). Some individual knock-out mice had hematocrits that appeared to be as low as 18%. The knock-out mice with basophilic macrocytes and anemia appeared to have marked splenomegaly on autopsy. Some spleens also appeared to show evidence of infarctions. Examination of cytospin preparations of spleen cells and spleen sections of such mice revealed an apparent nearly complete replacement of splenic lymphocytes by dysplastic, intensely basophilic erythroblasts. In some cases, extrameduallary erythropoiesis in the liver appeared to result in the formation of pedunculated hepatic tumors. Analysis of the knock-out mice created in Example 7 indicates that the Penumbra gene appears to play a role in normal erythropoiesis as the mice appear to develop dyserythropoiesis, anemia and splenomegaly

### Example 9

This example describes involvement of Penumbra gene products in erythropoiesis. A continuous multipotent hematopoietic cell line, EMX (for “Erythroid-Myeloid-and-Unknown”), was established from the bone marrow of a knock-out mouse created in Example 7. A clonal derivative, EMX C.1, was then used as the prototype and maintained with SCF, thrombopoietin (TPO) and interleukin-3 (IL-3). In the presence of SCF+TPO+IL-3 plus EPO, EMX C.1 appeared to differentiate into erythrocytes, monocytes, neutrophils, mast cells and megakaryocytes. Genotyping confirmed that EMX C.1 was deficient in the Penumbra gene. Although EMX C.1 appeared capable of differentiating into erythrocytes, the efficiency was low as reflected in the low levels of GATA-1 and β^{major}-globin mRNAs. Similar findings were seen in EMX C.1 transduced with the negative control retroviral vector MSCV-PGK-EGFP (“EMX/EGFP”) without or with EPO stimulation. In contrast, EMX C. 1 transduced with MSCV-Pen-PGK-EGFP (“E/Pen”, expressing mPen) exhibited robust erythropoiesis in response to erythropoietin (EPO) as evidenced by higher levels of GATA-1 and β^{major}-globin mRNAs. These findings indicate that Penumbra nucleic acids and proteins enhance erythropoiesis in EMX C.1 cells. Examination of Wright-Giemsa-stained cytospins of EMX/EGFP and EMX/Pen confirmed that after 12 days' stimulation with EPO (4 unit/ml), about 45% of the EMX/Pen cells were erythroblasts compared with only 5% in the control EMX/EGFP cultures.

### Example 10

This example describes an analysis of the levels of erythropoiesis via quantitative RT-PCR in both EMX/EGFP and EMX/Pen cell lines. Without added EPO, EMX/EGFP and EMX/Pen cells expressed similar levels of EPO receptor (EPO-R) mRNA. However, the expression of β^{major}-globin mRNA in the EMX/Pen cells was seven-fold higher than in the EMX/EGFP cells. The expression of β^{major}-globin mRNA by EMX/Pen in the absence of added EPO was probably stimulated by the trace EPO in Fetal Bovine Serum (FBS). Addition of EPO (4 unit/ml) for 6-10 days greatly stimulated β^{major}-globin expression in EMX/Pen but appeared to have only a modest effect on EMX/EGFP. This indicates that Penumbra appears to play an important role in the survival, proliferation and/or differentiation of EPO-responsive erythroid progenitors.

### Example 11

This example describes identification of the developmental stages at which erythropoiesis was affected by deletion of the Penumbra gene. EMX/EGFP or EMX/Pen that had been stimulated with EPO (4 unit/ml) in addition to SCF+TPO+IL-3 for 14 days were washed free of cytokines and re-cultured in a medium containing EPO alone at various concentrations. All non-erythroid progenitors died due to lack of relevant growth factors. Only EPO-responsive progenitors survived the switch in culture medium. Cells surviving the EPO-alone switch were enumerated or subjected to colony assays in the presence of SCF+IL-3+EPO. While very few EMX C.1 or EMX/EGFP cells survived the EPO-alone switch, many EMX/Pen cells did survive. These EPO-rescued EMX/Pen cells appeared to consist of erythroblasts at all stages of differentiation as well as undifferentiated blasts. Clonogenic assays of EPO-rescued cells from the EMX/Pen cultures appeared to reveal large numbers of large, medium and small burst-forming unit-erythroid (BFU-E)-derived colonies. In contrast, very few BFU-E-derived colonies appeared to be found in the control EMX/EGFP cultures. This indicates that Pen^{-/-} (knock-out) BFU-E are hypo-responsive to EPO and that this defect is complemented by replacement of Penumbra products.

All of the COMPOSITIONS, METHODS and APPARATUS disclosed and claimed herein can be made and executed without undue experimentation in light of the present disclosure. While the compositions and methods of this invention have been described in terms of particular embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-20. (canceled)

21. An isolated nucleic acid molecule having a nucleic acid sequence selected from the group consisting of SEQ ID NO:1, SEQ ID NO:2, SEQ ID NO:4, SEQ ID NO:6, SEQ ID NO:7, SEQ ID NO:8, and SEQ ID NO:9 and variants thereof that have at least 60% sequence homology to a nucleic acid sequence selected from the group consisting of SEQ ID NO: 1, SEQ ID NO:2, SEQ ID NO:4, SEQ ID NO:6, SEQ ID NO:7, SEQ ID NO:8, and SEQ ID NO:9.

22. The isolated nucleic acid molecule of claim 21, wherein said nucleic acid molecule is included in a recombinant molecule selected from the group consisting of recombinant viruses, recombinant vectors, and recombinant cells.

23. The isolated nucleic acid molecule of claim 22, wherein said recombinant cell comprises a host cell transformed with one or more recombinant molecules.

24. The isolated nucleic acid molecule of claim 23, wherein said recombinant cell is EMX.

25. The isolated nucleic acid molecule of claim 21, wherein said nucleic acid molecule encodes a Penumbra protein.

26. The isolated nucleic acid molecule of claim 21 that further encodes a protein having an amino acid sequence selected from the group consisting of SEQ ID NO:3 and SEQ ID NO:5 and proteins 90% identical to the amino acid sequence of SEQ ID NO:3 and SEQ ID NO:5.

27. The isolated nucleic acid molecule of claim 26, wherein said protein is administered to cells in order to increase said cell responsiveness to erythropoietin.

28. The isolated nucleic acid molecule of claim 26, wherein said protein is used in the treatment of a disease selected from the group consisting of acute myelogenous leukemias, myeloproliferative disorders, chronic myelogenous leukemia, juvenile myelomonocytic leukemia, transient myeloproliferative disorder and myelodysplastic syndrome.

29. The isolated nucleic acid molecule of claim 21, wherein said isolated nucleic acid molecule is introduced into a cell in an amount effective to increase said erythropoietin responsiveness of said cell.

30. A method of detecting 7q31q32-related deletions in myeloid malignancies comprising conducting a hybridization reaction with a nucleic acid probe and a sample; detecting the presence of hybridization; and analyzing the results to determine the presence or absence of a deletion.

31. The method of claim 30, wherein said probe is selected from the group consisting of SEQ ID NO: 1, SEQ ID NO:2, SEQ ID NO:4, SEQ ID NO:6, SEQ ID NO:7, SEQ ID NO:8, and SEQ ID NO:9.

32. The method of claim 30, wherein said myeloid malignancy is selected from the group consisting of acute myelogenous leukemia, myeloproliferative disorders, chronic myelogenous leukemia, juvenile myelomonocytic leukemia, transient myeloproliferative disorder and myelodysplastic syndromes.

33. The method of claim 30, wherein said detecting 7q31q32-related deletions in myeloid malignancies occurs on a biochip.

34. A method of treating a myeloid malignancy in an individual comprising administering to said individual a therapeutically effective amount of a Penumbra protein.

35. The method of claim 34, wherein said Penumbra protein is sequence selected from the group consisting of SEQ ID NO:3 and SEQ ID NO:5.

36. The method of claim 35, wherein said Penumbra protein increases erythropoietin responsiveness in said individual.

37. The method of claim 34 wherein said myeloid malignancy is selected from the group consisting of acute myelogenous leukemia, myeloproliferative disorders, chronic myelogenous leukemia, juvenile myelomonocytic leukemia, transient myeloproliferative disorder and myelodysplastic syndromes.
